(19) United States
(12) Patent Application Publication
(10) Pub. No.: US 2009/0227547 A1
(43) Pub. Date: Sep. 10, 2009

(54) Novel salt form of a Beta2-adrenergic agonist quinolin-2-one derivative

(75) Inventors: William Patrick Martin, Kent (GB); Nell Michael Smith, Hertfordshire (GB); Lois Elizabeth Vernon, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited, Greenford, Middlesex, EN (GB)

(21) Appl. No.: 11/917,190

(22) PCT Filed: Jun. 13, 2006
(86) PCT No.: PCT/EP06/05763

§ 371 Date: Oct. 1, 2008

(30) Foreign Application Priority Data
Jun. 15, 2005 (GB) 0512246.0

(51) Int. Cl. A61K 31/568; C07D 215/227; A61K 31/4704

(52) U.S. Cl. 514/171; 546/157; 514/312

(57) ABSTRACT

A 2,5-dichlorobenzenesulfonate salt of compound (I):

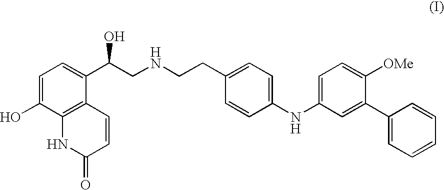

is disclosed along with formulations thereof, processes for making the same, and methods of treatment or prophylaxis administering the same.

The present invention relates to a novel pharmaceutical, to a process for the preparation of said pharmaceutical and to the use of said pharmaceutical in medicine. In particular this invention relates to a novel salt of a β₂ adrenergic agonist.

β₂ Adrenergic receptor agonists are recognised as effective drugs for the treatment of pulmonary diseases such as asthma and chronic obstructive pulmonary disease (including chronic bronchitis and emphysema). β₂ Adrenergic receptor agonists are also recognised as useful for treating premature labour, and are potentially useful for treating neurological disorders and cardiac disorders.

International Patent Application WO 03/042164 (Theravance Inc) relates to novel compounds described as having β₂ adrenergic receptor activity. Inter alia, International Patent Application WO 03/042164 describes compounds of the formula (I):

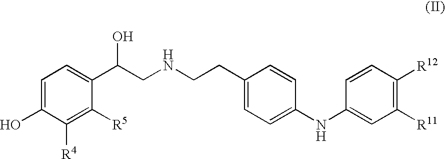

wherein:
R⁴ is —CH₂OH or —NHCHO and R⁵ is hydrogen; or R⁴ and R⁵ taken together are —NHC(═O)CH═CH—;
R¹¹ is phenyl or heteroaryl, wherein each phenyl is optionally substituted with 1 or 2 sustituents selected from halo, —OR^{d}, —CN, —NO₂, —SO₂R^{d}, —C(═O)R^{d}, —C(═O)NR^{d}R^{e}, and C₁₋₃alkyl, wherein C₁₋₃alkyl is optionally substituted with 1 or 2 substituents selected from carboxy, hydroxy, and amino, and each R^{d} and R^{e} is independently hydrogen or C₁₋₃alkyl; and wherein each heteroaryl is optionally substituted with 1 or 2 C₁₋₃ alkyl substituents; and R¹² is hydrogen or —OC₁₋₆ alkyl;
or a pharmaceutically acceptable salt or solvate or stereoisomer thereof.

The compound of Example 61 of International Patent Application WO 03/042164 is:

N-{2-[4-(3-phenyl-4-methoxyphenyl)aminophenyl]ethyl}-(R)-2-hydroxy-2-(8-hydroxy-2(1H)-quinolinon-5-yl)ethylamine which may be represented by the structural formula (I):

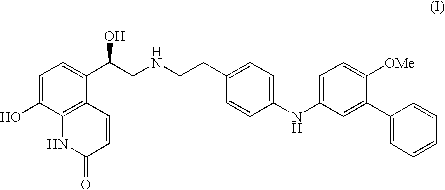

hereinafter also referred to as “Compound (I)”.

WO 2004/101525 (Theravance Inc.) relates to crystalline monohydrochloride salt of Compound (I) in solvate form.

We have now found that Compound (I) forms a novel 2,5-dichlorobenzenesulfonate salt (hereinafter also referred to as the ‘2,5-Dichlorobenzenesulfonate’).

Said salt has demonstrated a range of useful properties. Thus in general it has good physical and chemical stability on storage.

Said salt has been shown to be substantially non-hygroscopic and to be non-solvated.

Said salt can be prepared in crystalline form.

In a first aspect therefore, the present invention provides a 2,5-dichlorobenzenesulfonate salt of Compound (I) viz, N-{2-[4-(3-phenyl-4-methoxyphenyl)aminophenyl]ethyl}-(R)-2-hydroxy-2-(8-hydroxy-2(1H)-quinolinon-5-yl)ethylammonium 2,5-dichlorobenzenesulfonate. The chemical name of compound (I) 2,5-dichlorobenzenesulfonate may also be represented as: N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate.

In one embodiment, the ratio of Compound (I) to dichlorobenzenesulfonic acid (by mole) is 1:1.

The invention also provides N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate in crystalline form.

The invention also provides crystalline N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate characterised by an X-ray powder diffraction (XRPD) pattern including diffraction peaks at the following 2θ values: 11.6±0.1; 14.5±0.1; 23.2±0.1; 25.0±0.1; 27±0.1.

In a further embodiment, the invention provides N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate characterised by an X-ray powder diffraction (XRPD) pattern substantially in accordance with FIG. 1.

In a further embodiment, the invention provides N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate characterised by an X-ray powder diffraction (XRPD) pattern substantially in accordance with FIG. 3.

In yet a further embodiment of the invention there is provided N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate characterised in that it has a melting point onset measured by DSC (0.5° C.) in the range of 190-230° C., for example in the range 219-223° C.

The present invention also provides a process for preparing N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate, characterised in that Compound (I) or a salt thereof is contacted with a source of 2,5-dichlorobenzenesulfonate ion and the 2,5-Dichlorobenzenesulfonate is recovered.

Compound (I) may be used in the reaction as a free base or in a protonated form. Thus, a salt of Compound (I) may be employed in the reaction, for example the acetate salt.

Compound (I) or a salt thereof may be dispersed or dissolved in a suitable solvent prior to contacting said compound with a source of 2,5-dichlorobenzenesulfonate ion. When compound (I) is employed as the free base, the reaction may for example be effected in a solvent such as dimethylsulphoxide, N,N-dimethylpyrrolidone, N,N-dimethylformamide or N,N-dimethylacetamide. In general it is desirable that the free base has good solubility and stability in the solvent used. However, the reaction may also be effected in other solvents such as ethanol. When it is desired to use a protonated form of Compound (I) this may be generated using an acid, for example, acetic acid, in a solvent such as those listed above, or in a lower alcohol, for example methanol, ethanol or isopropanol; or tetrahydrofuran.

In one embodiment Compound (I) or a salt thereof may be generated from a protected form of Compound (I) (for example as described hereinafter) and the product used directly in preparation of the 2,5-Dichlorobenzenesulfonate.

The source of 2,5-dichlorobenzenesulfonate ion may be 2,5-dichlorobenzenesulfonic acid, for example in the form of a hydrate e.g. the dihydrate. The 2,5-dichlorobenzenesulfonic acid may be used in solution or as a solid. Solvents for the dichlorobenzenesulfonic acid include water, or a lower alcohol such as methanol or ethanol, or a mixture of such solvents.

The reaction may be effected at ambient temperature or at an elevated temperature, for example at the reflux temperature of the solvent, although any convenient temperature that provides the required product may be employed.

Recovery of the required compound may comprise crystallisation from an appropriate solvent, conveniently the reaction solvent, for example by cooling, seeding, or use of an antisolvent. Thus, for example, the 2,5-Dichlorobenzenesulfonate may be crystallised from a solvent such as dimethylsulfoxide, by addition of an anti-solvent. Solvents for Compound (I) 2,5-dichlorobenzenesulfonate include dimethylsulphoxide, N,N-dimethylpyrrolidone, N,N-dimethylformamide and N,N-dimethylacetamide. We have found said salt has limited solubility and therefore a wide range of solvents can be employed as antisolvents, including water, tetrahydrofuran, lower alcohols, e.g. methanol, acetone, and the like. When the reaction medium comprises an acid, such as acetic acid, in a solvent such as a lower alcohol, for example methanol, ethanol or isopropanol; or tetrahydrofuran, the 2,5-Dichlorobenzenesulphonate salt may crystallise spontaneously following reaction of the protonated compound of formula (I) with a source of 2,5-dichlorobenzenesulphonate ion.

The 2,5-Dichlorobenzenesulfonate may if desired or necessary be recrystallised for example by cooling, or use of an antisolvent. Thus for example the 2,5-Dichlorobenzenesulfonate may be recrystallised by dissolving the compound in an appropriate solvent for example, dimethylsulphoxide, N,N-dimethylpyrrolidone, N,N-dimethylformamide, N,N-dimethylacetamide or aqueous tetrahydrofuran and contacting said solution with an anti-solvent. Exemplary antisolvents include water, tetrahydrofuran, lower alcohols, e.g. methanol, acetone, and the like.

When using aqueous tetrahydrofuran as the appropriate solvent we have found that the 2,5-Dichlorobenzenesulphonate can be dissolved in a tetrahydrofuran:water mixture, having a composition of, for example, 80-70% tetrahydrofuran: 20-30% water. Crystallisation may be initiated by adjusting the relative amounts of tetrahydrofuran and water, for example by addition of further water or further tetrahydrofuran, or by distilling tetrahydrofuran from the mixture. Thus for example, crystallisation may be initiated by adjusting the composition in the range 60-30% tetrahydrofuran:40-70% water, such as 50-60% tetrahydrofuran:50-40% water, or 30-40% tetrahydrofuran:70-60% water. It will however be apparent to the skilled worker that because both dissolution and crystallization are affected by a range of factors, for example temperature, the aforementioned ranges should not be regarded as absolute limits. On the basis of the teachings herein the skilled person can readily determine the solubility of the 2,5-Dichlorobenzesulphonate in a range of solvent mixtures and hence determine suitable relative compositions for both dissolving and recrystallising the 2,5-Dichlorobenzenesulphonate, without undue experimentation. Optimal compositions may for example depend on the total volume of solvent used.

In either the initial recovery of the crystalline material or a subsequent recrystallisation, crystallisation may be assisted by cooling, for example in the range 1-10° C.

Crystallisation may also be initiated by seeding with crystals of the 2,5-Dichlorobenzenesulfonate.

Compound (I) may be prepared for example, by the general methods described in WO03/042164 and WO 2004/101525, for example according to the following route:

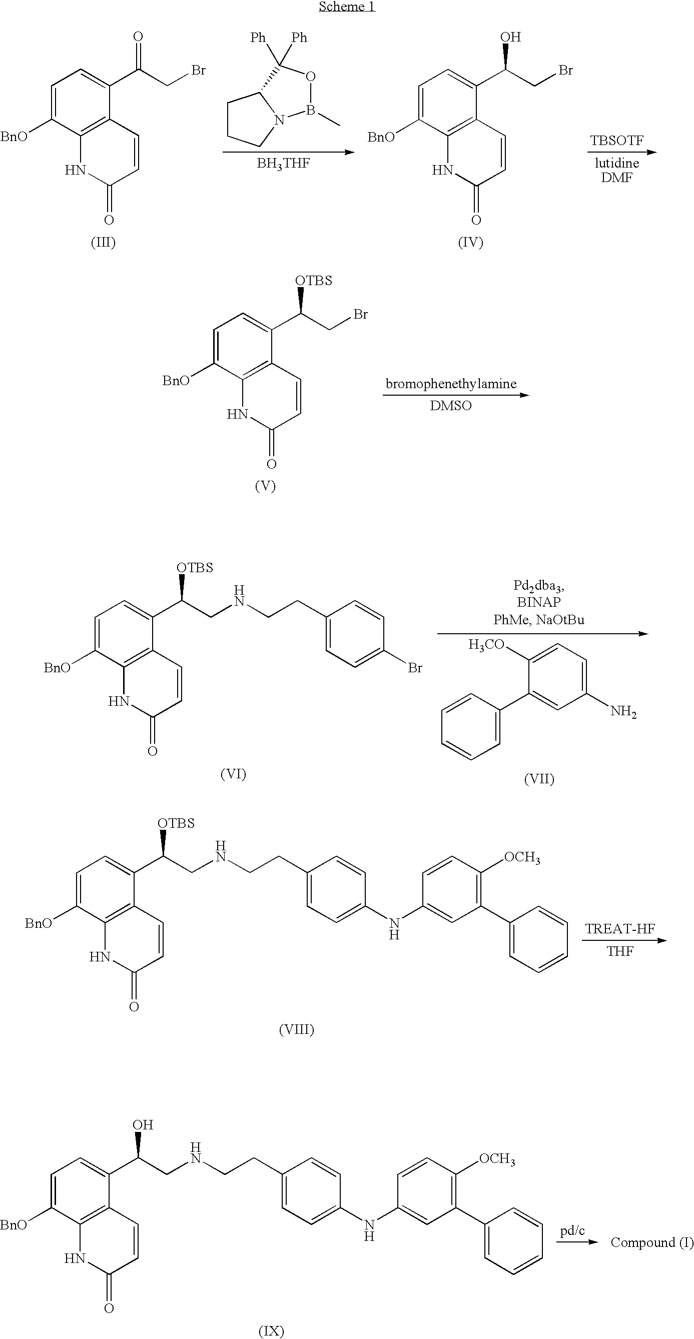

In Scheme 1 the abbreviations used have the following meanings:

Bn—benzyl
Ph—phenyl
TBS—tert-butyldimethylsilyl
TBSOTF—tert-butyldimethylsilyl trifluoromethane sulfonate
Pd₂ dba₃-tris(dibenzylideneacetone)dipalladium(0)
BINAP—2,2′-bis(diphenylphosphino)-1,1′-binaphthyl
DMF—dimethylformamide
DMSO—dimethylsulfoxide
THF—tetrahydrofuran
TREAT-HF—triethylamine trihydrofluoride

It will be appreciated that while specific process conditions (i.e. reaction temperatures, times, mole ratios of reactants, solvents, pressures, etc.) are given, other process conditions can also be used unless otherwise stated. Likewise, protecting groups other than those depicted in Scheme 1 may be employed. Selection of appropriate protecting groups is within the remit of the skilled chemist and may be achieved without undue experimentation.

Intermediate (III), 5-(2-bromo-1-oxy)ethyl-8-benzyloxy-2(1H)-quinolinone, may be prepared as described in EP147791B. Intermediate (IV), 5-(2-bromo-(R)-1-hydroxy)ethyl-8-benzyloxy-2(1H)-quinolinone, may be formed by the chiral reduction of intermediate (III) using an oxazaborolidine catalyst, prepared in situ following a procedure described in Mathre et al. J. Org. Chem., 1991, 56, 751-762 The protected Intermediate (V), 5-(2-bromo-(R)-1-tert-butyldimethylsiloxy)ethyl-8-benzyloxy-2(1H)-quinolinone, may be formed by the addition of tert-butyldimethylsilyl trifluoromethane sulfonate (TBSOTF) and lutidine to intermediate (IV), dissolved in dimethylformamide (DMF).

Intermediate (VI), N-[2-(4-bromophenyl)ethyl]-(R)-2-tert-butyldimethylsiloxy-2-(8-benzyloxy-2(1H)-quinolinon-5-yl)ethylamine, may obtained as the solid hydrochloride salt by reaction of (V) with 4-bromophenethylamine. Intermediate (VI) may be coupled with 4-methoxy 3-phenylaniline hydrochloride (VII) in the presence of a catalyst comprising 2,2′-bis(diphenylphosphino)-1,1′-binaphthyl and tris(dibenzylideneacetone)dipalladium(0) to give the protected diarylamine Intermediate (VII), N-{2-[4-(3-phenyl-4-methoxyphenyl)aminophenyl]ethyl}-(R)-2-tert-butyldimethylsilyl-2-(8-benzyloxy-2(H)-quinolinon-5-yl)ethylamine.

Alternatively, a protected intermediate of formula (VIII) may be obtained by the reaction of intermediate of formula (V) above with a compound of formula (X):

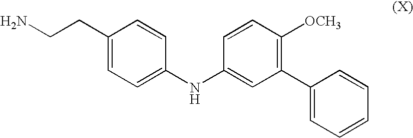

or a salt thereof, e.g. the dihydrobromide.

The reaction may be effected in a solvent such as toluene and in the presence of a base such as potassium carbonate.

A compound of formula (X) may be prepared by a coupling reaction of a compound of formula (XI)

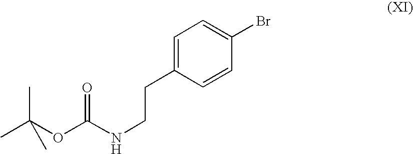

with a compound of formula (VII)

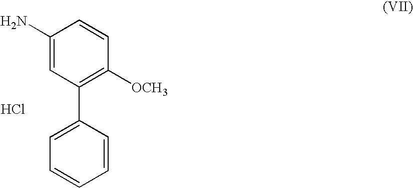

for example in the form of the hydrochloride, in the presence of a catalyst such as palladium acetate, PdCl₂, Pd(PPh₃)₄, Pd(dba)₂ or Pd₂(dba)₃; and a phosphine such as triphenylphosphine, (di-tert-butylphosphino)biphenyl, tricyclohexylphosphine, triisopropylphosphine, tricyclopentylphosphine, or tri-tert-butylphosphine; and a base such as aqueous potassium or sodium phosphate, potassium or sodium carbonate, sodium acetate or sodium tert-butoxide (NaOtBu) in a suitable solvent such as toluene; followed by removal of the protecting group, using for example isopropyl alcohol and hydrogen bromide in a suitable solvent, for example, toluene.

The compound of formula (XI) may be prepared from a compound of formula (XII)

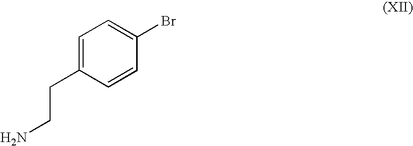

using, for example, di tert-butyl dicarbonate (Boc₂O) and dichloromethane (DCM). It will be appreciated that the compound of formula (XI) may be formed with alternative protecting groups in place of the tert-butoxycarbonyl group. Thus for example a benzyl carbamate protecting group may be introduced using benzyl chloroformate in dichloromethane in the presence of Hunig's base or a diphenyloxazolone protecting group may be introduced using 4,5-diphenyl-1,3-dioxol-2-one in dichloromethane.

The TBS protecting group may be removed from Intermediate (VIII) by addition of triethylamine trihydrofluoride (TREAT HF) in tetrahydrofuran or hydrochloric acid in methanol, giving Intermediate (IX), N-{2-[4-(3-phenyl-4-methoxyphenyl)aminophenyl]ethyl}-(R)-2-hydroxy-2-(8-benzyloxy-2(1H)-quinolinon-5-yl)ethylamine.

The benzyl protecting group may be removed from the compound of formula (IX) by conventional means, for example by hydrogenolysis using palladium on activated carbon. Compound (I) may conveniently be generated in situ and further reacted without isolation. Thus the aforementioned deprotection may be effected in a solvent which is also suitable for the step of forming the 2,5-dichlorobenzenesulfonate salt, as described hereinabove. We have also found that when Compound (I) is generated in situ, it is advantageously in protonated form, for example, as an acetate. Thus, for example, deprotection may be effected in methanol and glacial acetic acid, to give Compound (I) in protonated form, which may be employed directly to form the 2,5-Dichlorobenzenesulfonate, for example, by reaction with a source of 2,5-dichlorobenzenesulphonate ion in an aqueous medium. Thus in a further aspect of the invention there is provided a process for preparing a 2,5-dichlorobenzenesulphonate salt of Compound (I) which comprises deprotecting a protected form of Compound (I), generating a protonated (salt) form of Compound (I) and further reacting with a source of 2,5-dichlorobenzenesulphonate ion.

In a further aspect the present invention provides a method for the prophylaxis or treatment of a clinical condition in a mammal, such as a human, for which a 2-adrenoreceptor agonist is indicated, which comprises administration of a therapeutically effective amount of N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate. In particular, the present invention provides such a method for the prophylaxis or treatment of a disease associated with reversible airways obstruction such as asthma, chronic obstructive pulmonary disease (COPD), respiratory tract infection or upper respiratory tract disease.

In the alternative, there is also provided N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate for use in medical therapy, particularly, for use in the prophylaxis or treatment of a clinical condition in a mammal, such as a human, for which a β₂-adrenoreceptor agonist is indicated. In particular, there is provided N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate for the prophylaxis or treatment of a disease associated with reversible airways obstruction such as asthma, chronic obstructive pulmonary disease (COPD), respiratory tract infection or upper respiratory tract disease.

The present invention also provides the use of N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate in the manufacture of a medicament for the prophylaxis or treatment of a clinical condition for which a 2-adrenoreceptor agonist is indicated, for example a disease associated with reversible airways obstruction such as asthma, chronic obstructive pulmonary disease (COPD), respiratory tract infection or upper respiratory tract disease.

The amount of 2,5-Dichlorobenzenesulfonate which is required to achieve a therapeutic effect will, of course, vary with the route of administration, the subject under treatment, and the particular disorder or disease being treated. The compound of the invention may be administered by inhalation at a dose of from 0.005 mg to 10 mg, preferably from 0.01 mg to 5.0 mg. e.g. from 0.1 mg to 2.5 mg. The dose range for adult humans may be from 0.005 mg to 10 mg per day, e.g. from 0.01 mg to 5.0 mg per day, conveniently from 0.1 mg to 2.5 mg per day, e.g. from 0.5 mg to 1.5 mg

While it is possible for the 2,5-Dichlorobenzenesulfonate to be administered alone, it is preferable to present it as a pharmaceutical formulation.

Accordingly, the present invention further provides a pharmaceutical formulation comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate and a pharmaceutically acceptable carrier or excipient, and optionally one or more other therapeutic ingredients.

Hereinafter, the term “active ingredient” means N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate, unless the context dictates otherwise.

The formulations include those suitable for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di or poly-saccharides (e.g. lactose or starch). Use of lactose is preferred. Powder blend formulations may also contain a ternary agent such as a sugar ester, for example cellobiose octaacetate, or a stearate such as magnesium stearate or calcium stearate.

Each capsule or cartridge may generally contain between 20 μg-10 mg of the active ingredient optionally in combination with another therapeutically active ingredient. Alternatively, the compound of the invention may be presented without excipients. Packaging of the formulation may be suitable for unit dose or multi-dose delivery. In the case of multi-dose delivery, the formulation can be pre-metered (e.g. as in Diskus, see GB 2242134, U.S. Pat. Nos. 6,632,666, 5,860,419, 5,873,360 and 5,590,645 or Diskhaler, see GB 2178965, 2129691 and 2169265, U.S. Pat. Nos. 4,778,054, 4,811,731, 5,035,237) or metered in use (e.g. as in Turbuhaler, see EP 69715 or in the devices described in U.S. Pat. No. 6,321,747). An example of a unit-dose device is Rotahaler (see GB 2064336 and U.S. Pat. No. 4,353,656). The Diskus inhalation device comprises an elongate strip formed from a base sheet having a plurality of recesses spaced along its length and a lid sheet hermetically but peelably sealed thereto to define a plurality of containers, each container having therein an inhalable formulation containing active ingredient which may be combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet. Alternatively, the formulation may be presented if desired together with one or more other therapeutic agents in an inhalation device wherein the individual therapeutic agents are administrable simultaneously but are stored separately (or wholly or partly stored separately for triple combinations), e.g. in separate pharmaceutical compositions, for example as described in WO 03/061743.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the active ingredient optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants e.g. oleic acid, sodium trioleate or lecithin and cosolvents e.g. ethanol. Pressurised formulations will generally be retained in a canister (e.g. an aluminium canister) closed with a valve (e.g. a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means e.g. by micronisation. The desired fraction may be separated out by air classification or sieving. Preferably, the particles will be crystalline. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD (mass median diameter) of 60-90 μm and not more than 15% will have a MMD of less than 15 μm.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

The compound and pharmaceutical formulations according to the invention may be used in combination with or include one or more other therapeutic agents, for example selected from anti-inflammatory agents, anticholinergic agents (particularly an M₁, M₂, M₁/M₂ or M₃ receptor antagonist), other β₂-adrenoreceptor agonists, antiinfective agents (e.g. antibiotics, antivirals), or antihistamines. The invention thus provides, in a further aspect, a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with one or more other therapeutically active agents, for example selected from an anti-inflammatory agent (for example a corticosteroid or an NSAID), an anticholinergic agent, another β₂-adrenoreceptor agonist, an antiinfective agent (e.g. an antibiotic or an antiviral), or an antihistamine. In one embodiment the invention provides combinations comprising 2,5-Dichlorobenzenesulfonate together with a corticosteroid, and/or an anticholinergic, and/or a PDE-4 inhibitor. In further embodiment the invention provides combinations comprising 2,5-Dichlorobenzenesulfonate together with one or two of said other therapeutic agents.

It will be clear to a person skilled in the art that, where appropriate, the other therapeutic ingredient(s) may be used in the form of salts, (e.g. as alkali metal or amine salts or as acid addition salts), or prodrugs, or as esters (e.g. lower alkyl esters), or as solvates (e.g. hydrates) to optimise the activity and/or stability and/or physical characteristics (e.g. solubility) of the therapeutic ingredient. It will be clear also that where appropriate, the therapeutic ingredients may be used in optically pure form.

Anti-inflammatory agents include corticosteroids. Exemplary corticosteroids include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester, 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. Preferred corticosteroids include fluticasone propionate, 6α,9β-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, in particular 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Non-steroidal compounds having glucocorticoid agonism that may possess selectivity for transrepression over transactivation and that may be useful in combination therapy include those covered in the following patents: WO03/082827, WO01/10143, WO98/54159, WO04/005229, WO04/009016, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277.

Anti-inflammatory agents include non-steroidal anti-inflammatory drugs (NSAID's).

Exemplary NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example, montelukast), iNOS inhibitors, tryptase and elastase inhibitors, beta-2 integrin antagonists and adenosine receptor agonists or antagonists (for example, adenosine 2a agonists), cytokine antagonists (for example, chemokine antagonists, such as a CCR3 antagonist) or inhibitors of cytokine synthesis, or 5-lipoxygenase inhibitors. iNOS inhibitors include those disclosed in WO93/13055, WO98/30537, WO02/50021, WO95/34534 and WO99/62875. CCR3 inhibitors include those disclosed in WO02/26722. Adenosine 2a agonists include those disclosed in WO05/116037.

A PDE4-specific inhibitor may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds of interest include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Another compound of interest is cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438. Further compounds of interest are disclosed in the published international patent application WO04/024728 (Glaxo Group Ltd), PCT/EP2003/014867 (Glaxo Group Ltd) and PCT/EP2004/005494 (Glaxo Group Ltd).

Anticholinergic agents of interest are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva).

Other anticholinergic agents include those described in WO2004/091482, WO2005/009439, WO2005/009362, WO2005/009440, PCT/US2004/033638, PCT/US2004/034234, PCT/US2004/036663, PCT/US2004/040667, PCT/US2004/040668, PCT/US2004/001333, PCT/US2004/08032, PCT/US2004/08026, PCT/US2004/08025, and PCT/US2004/08027.

Antihistamines of interest (also referred to as H1-receptor antagonists) include any one or more of the numerous antagonists known which inhibit H1-receptors, and are safe for human use. First generation antagonists, include derivatives of ethanolamines, ethylenediamines, and alkylamines, such as diphenylhydramine, pyrilamine, clemastine, chlorpheniramine. Second generation antagonists, which are non-sedating, include loratidine, desloratidine, terfenadine, astemizole, acrivastine, azelastine, levocetirizine fexofenadine and cetirizine and efletirizine, especially cetirizine, levocetirizine, efletirizine and fexofenadine. Other histamine receptor antagonists which may be used alone, or in combination with an H1 receptor antagonist include antagonists (and/or inverse agonists) of the H3 receptor, for example, the compounds disclosed in WO04/035556, and antagonists (and/or inverse agonists) of the H4 receptor.

The invention thus provides, in a further aspect, a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with a PDE4 inhibitor. For example, the invention provides a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with a PDE4 inhibitor as specified hereinabove, e.g. cis-4-cyano-4-[3-(cyclopentyloxy)-4-methoxyphenyl]cyclohexane-1-carboxylic acid.

The invention thus provides, in a further aspect, a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with a corticosteroid. For example, the invention provides a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with a specific corticosteroid as described hereinabove, e.g. fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

The invention thus provides, in a further aspect, a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with an anticholinergic. For example, the invention provides a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with a specific anticholinergic as described hereinabove, e.g. ipratropium, oxitropium or tiotropium

The invention thus provides, in a further aspect, a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with an antihistamine. For example, the invention provides a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with a specific antihistamine as described hereinabove.

The invention thus provides, in a further aspect, a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with a PDE4 inhibitor and a corticosteroid. For example, the invention provides a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with a specific antihistamine and a specific corticosteroid as described hereinabove.

The invention thus provides, in a further aspect, a combination comprising N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with an anticholinergic and a PDE-4 inhibitor. For example, the invention provides a combination N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate together with a specific PDE4 inhibitor and a specific anticholinergic as described hereinabove.

The combinations referred to above may conveniently be presented for use in the form of a pharmaceutical formulation and thus pharmaceutical formulations comprising a combination as defined above together with a physiologically acceptable diluent or carrier represent a further aspect of the invention.

The individual compounds of such combinations may be administered either sequentially or simultaneously in separate or combined pharmaceutical formulations. Appropriate doses of known therapeutic agents will be readily appreciated by those skilled in the art.

### BRIEF DESCRIPTION OF THE DRAWINGS

FIG. 1 shows an X-ray powder diffraction pattern of N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate; corresponding to Example 3.

FIG. 2 shows a DSC trace of N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate; corresponding to Example 3. There are endotherms with onsets of 33° C. and 219° C. The first endotherm is due to the loss of solvent. This is followed by a larger endotherm due to degradation and melting with an onset of 219° C.

FIG. 3 shows an X-ray powder diffraction pattern of N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate; corresponding to Example 5

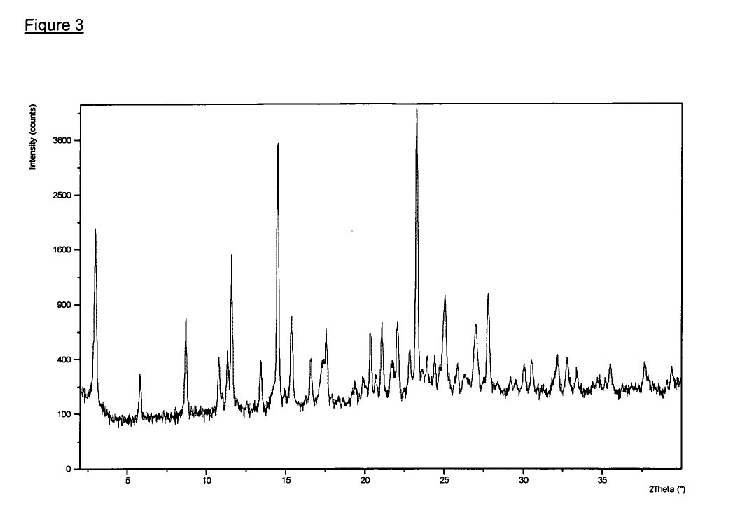

FIG. 4 shows the DSC trace of N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate; corresponding to Example 5.

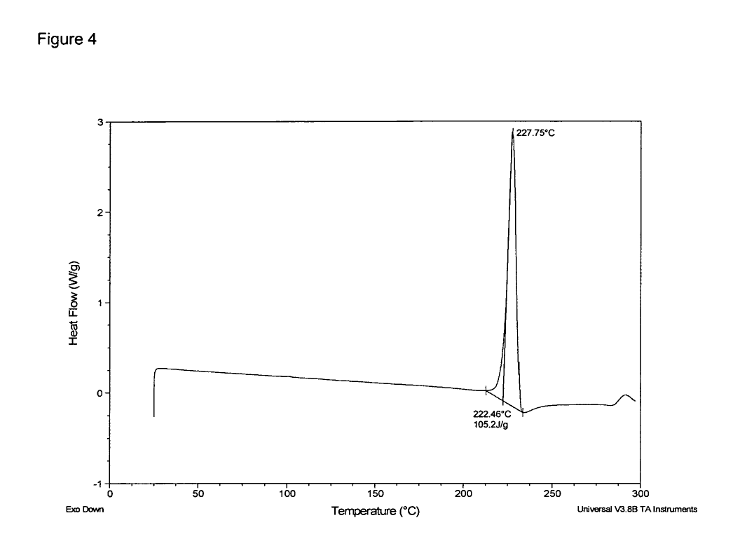

For a better understanding of the invention, the following Examples are given by way of illustration.

### Analytical Methods

### XRPD

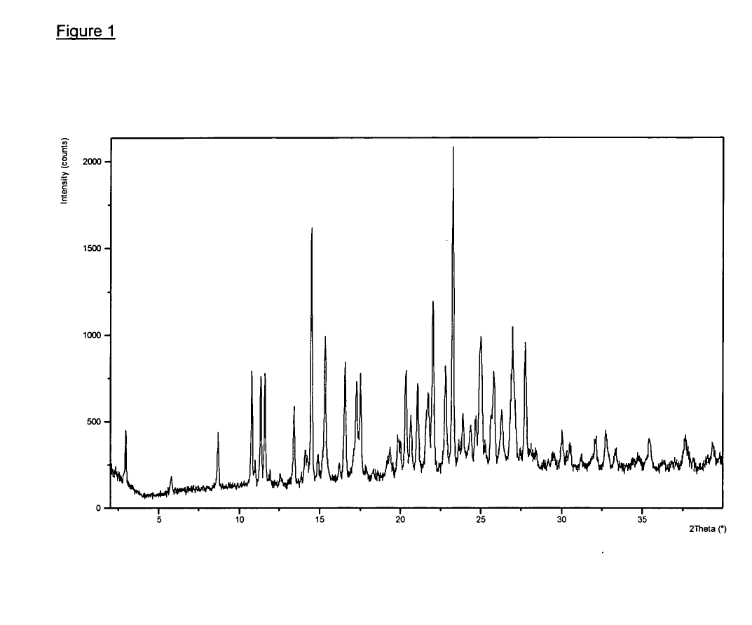

The X-ray powder diffraction (XRPD) analysis shown in FIG. 1 was performed on a PANalytical X'Pert Pro powder diffractometer, model PW3040/60, serial number DY1850 using an X'Celerator detector. The acquisition conditions were: radiation: Cu Kα, generator tension: 40 kV, generator current 45 mA, start angle: 2.0° 2θ, end angle 40° 2θ, step size 0.017° 2θ, time per step: 32 seconds. The sample was prepared as a thin layer of powder on a silicon wafer. Characteristic XRPD angles and d-spacings are recorded in Table 1.

### DSC

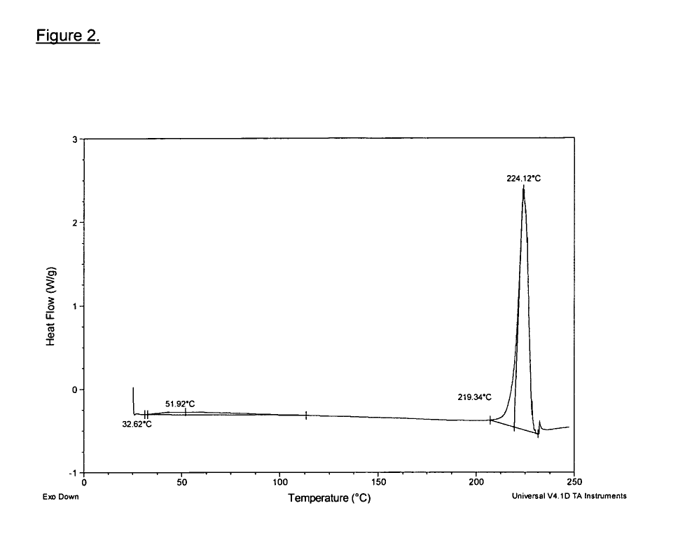

The DSC trace shown in FIG. 2 was obtained using a TA Instruments Q1000 calorimeter. The sample was weighed into an aluminium pan, a pan lid placed on top and lightly crimped without sealing the pan. The experiment was conducted using a heating rate of 10° C. min−1.

### NMR

1H NMR spectra were acquired on a 400 MHz Bruker DPX400 spectrometer at 300K. [Bruker AV400 (086281)], with the exception of the spectra for Example 5 which was acquired on a 500 MHz Bruker DRX500 spectrometer at 300K (Biomax 086283). Sample was dissolved in dmso-d6 and chemical shifts were reported in ppm relative to the TMS signal at 0 ppm.

### Abbreviations

HPLC—High performance liquid chromatography

### EXAMPLE 1

### N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate

(i) N-[2-[4-[(3-Phenyl-4-methoxyphenyl)amino]phenyl]ethyl]-(R)-2-hydroxy-2-(8-benzyloxy-1,2-dihydro-2-oxoquinolin-5-yl)ethylamine (23.8 kg, 38.9 mol) was dissolved in methanol (47.6 L) and glacial acetic acid (47.6 L). The resultant solution was charged to a hydrogenation vessel, washed into the vessel with a mixture of acetic acid (11.9 L) and methanol (11.9 L) and stirred at 17-23° C. The vessel was purged with nitrogen then charged with 10% Pd/C (ca. 50% w/w water-wet paste, 2.38 kg). The vessel was purged with nitrogen and then hydrogen. Hydrogen was introduced to the vessel at about 1 atmosphere pressure and the suspension was stirred at ambient temperature for about 6 hrs. The reaction was checked by HPLC, and on completion the suspension was filtered. The filter was washed with methanol/acetic acid (1:1 v/v, 2×35.7 L). The filtrate and washings were combined for use at the next stage.

(ii) 2,5-dichlorobenzenesulfonic acid dihydrate solution in water (26.9 kg, from a stock solution of 50 kg of acid in 111 L of water, 31.8 mol) was added to the combined filtrate and washings above at 16-19.5° C. over about 1 hour. Crystallisation commenced about halfway through the addition. The resultant suspension was stirred for about 16.5 hours at ambient temperature. The suspension was filtered under nitrogen pressure, and the solid washed with methanol/acetic acid (1:1 v:v, 23.8 L) and methanol (2×47.6 L). The solid was dried under vacuum at 50-60° C. to give N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate as a light green solid (22.85 kg, 96%).

NMR: δ(ppm): 2.77-2.93 (2H) m; 3.08 (1H) m; 3.11-3.24 (3H) m; 3.71 (3H) s; 5.31 (1H) m; 6.14 (1H) broad res; 6.59 (1H) d, J=9.8 Hz; 6.95 (2H) d, J=8.6 Hz; 6.97-7.05 (4H) m; 7.07 (2H) d, J=8.6 Hz; 7.16 (1H) d, J=8.1 Hz; 7.31 (1H) m; 7.37-7.43 (4H) m; 7.46 (2H) d, J=8.3 Hz; 7.84 (1H) d, J=2.5 Hz; 7.91 (1H) s; 8.15 (1H) d, J=10.0 Hz; 8.59 (2H) broad res; 10.06-10.75 (1H) broad res; 10.49 (1H) s.

### EXAMPLES 2-5

### Recrystallisation of N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate

### EXAMPLE 2

N-[2-[4-[(3-Phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate (6 g, 8.0 mmol) and dimethylsulfoxide (30 ml) were added to a 100 ml round-bottomed flask. The mixture was stirred while heating to 55-65° C. until complete dissolution had occurred. Water (30 ml) was added over about 30 mins and crystallisation occurred. The suspension was stirred at 55-65° C. for about 15 mins, then cooled to 20-25° C. and stirred for 18 hrs. The suspension was filtered under vacuum, and the solid washed with water (2×12 ml) then dried under vacuum at 50-60° C. to give N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate as a beige solid (5.58 g, 93%).

### EXAMPLE 3

N-[2-[4-[(3-Phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate (1.80 kg, 2.4 mol) and dimethylsulfoxide (9.0 L) were added to a 20 L jacketed vessel. The mixture was stirred and water (3.6 L) was added to the stirred suspension. The stirred suspension was heated to 55-65° C. until complete dissolution had occurred. The solution was seeded with N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate (5.0 g, 6.7 mmol) and stirred for 10 mins. Water (5.4 L) was added over about 2.5 hrs and crystallisation occurred. The suspension was stirred at 55-65° C. for about 10 mins, then cooled to 20-25° C. The suspension was filtered under vacuum, and the solid washed with water (2×3.6 L) then dried under vacuum at 50-60° C. to give N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate as a beige solid (1.69 kg, 93.9%).

XRPD analysis of the product is shown in FIG. 1 and characteristic XRPD peak angles and d-spacings are given in Table 1 below. DSC analysis of the product is shown in FIG. 2.

### EXAMPLE 4

N-[2-[4-[(3-Phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate (1.80 kg, 2.4 mol) and dimethylsulfoxide (9.0 L) were added to a 20 L jacketed vessel. The mixture was stirred and water (3.6 L) was added to the stirred suspension. The stirred suspension was heated to 75-85° C. until complete dissolution had occurred. The solution was seeded with N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate (5.0 g, 6.7 mmol) and stirred for 10 mins. Water (5.4 L) was added over about 50 mins and crystallisation occurred. The suspension was stirred at 75-85° C. for about 10 mins, then cooled to 20-25° C. The suspension was stirred at 20-25° C. for about 1.5 hrs then filtered under vacuum, and the solid washed with water (2×3.6 L) then dried under vacuum at 50-60° C. to give N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate as a beige solid (1.72 kg, 95.6%).

NMR: δ(ppm): 2.77-2.93 (2H) m; 3.08 (1H) m; 3.11-3.23 (3H) m; 3.71 (3H) s; 5.31 (1H) m; 6.15 (1H) broad res; 6.59 (1H) d, J=9.8 Hz; 6.95 (2H) d, J=8.6 Hz; 6.97-7.05 (4H) m; 7.07 (2H) d, J=8.6 Hz; 7.16 (1H) d, J=8.3 Hz; 7.31 (1H) m; 7.37-7.43 (4H) m; 7.46 (2H) d, J=8.6 Hz; 7.84 (1H) d, J=2.5 Hz; 7.91 (1H) s; 8.15 (1H) d, J=10.0 Hz; 8.59 (2H) broad res; 9.99-10.95 (1H) broad res; 10.49 (1H) s.

### EXAMPLE 5

N-[2-[4-[(3-Phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate (799.9 g) was dissolved in a mixture of tetrahydrofuran (6160 mL) and water (2640 mL) at 49-55° C. The solution was clarified by filtration through a 5 μm filter and the filter washed with a mixture of tetrahydrofuran (560 mL) and water (240 mL). Water (2400 mL) was added to the combined filtrate and wash over approximately 15 minutes while maintaining a temperature of 49-55° C. The solution was then seeded with a suspension of N-[2-[4-[(3-Phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate (2.0 g) in tetrahydrofuran (40 mL). Further water (6400 mL) was added over approximately 3 hours while maintaining a temperature of 49-55° C. The resulting suspension was cooled to 1-7° C. over 2 hours and then stirred for approximately 1 hour at this temperature. The product was collected by filtration and washed sequentially with a mixture of tetrahydrofuran (1750 mL) and water (3050 mL) and then acetone (3×4800 mL). The product was then dried overnight at 50° C. in vacuo to give N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate as an off-white solid (687.5 g, 86%).

¹H NMR (DMSO-d₆) 6: (ref to TMS at 0 ppm): 2.86 (2H, m); 3.08 (1H, m); 3.16 (3H, m); 3.71 (3H, s); 5.31 (1H, d, J=9.3 Hz); 6.15 (1H, s); 6.59 (1H, d, J=10.1 Hz); 6.92-7.10 (8H, m); 7.16 (1H, d, J=8.2 Hz); 7.31 (1H, t, J=7.1 Hz); 7.36-7.43 (4H, m); 7.46 (2H, d, J=7.4 Hz); 7.84 (1H, d, J=2.0 Hz); 7.91 (1H, s); 8.15 (1H, d, J=10.1 Hz); 8.59 (2H, broad s); 10.42 (1H, broad s); 10.49 (1H, s).

TABLE 1
Characteristic XRPD peak angles and d-spacings

| Two Theta (deg)* | d-spacing (Å) |
|---|---|
| Two Theta (deg) | d-spacing |
| 3.0 | 29.9 |
| 5.8 | 15.3 |
| 8.7 | 10.2 |
| 10.8 | 8.2 |
| 11.0 | 8.1 |
| 11.3 | 7.8 |
| 11.6 | 7.6 |
| 13.4 | 6.6 |
| 14.5 | 6.1 |
| 15.3 | 5.8 |
| 16.6 | 5.4 |
| 17.3 | 5.1 |
| 17.5 | 5.1 |
| 20.3 | 4.4 |
| 21.1 | 4.2 |
| 21.6 | 4.1 |
| 21.7 | 4.1 |
| 22.0 | 4.0 |
| 22.8 | 3.9 |
| 23.3 | 3.8 |
| 25.0 | 3.6 |
| 25.7 | 3.5 |
| 25.8 | 3.5 |
| 27.0 | 3.3 |
| 27.8 | 3.2 |

*For copper Kα radiation.

What is claimed is:

1. N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzene sulfonate.

2. A 2,5-dichlorobenzenesulfonate salt of compound (I):

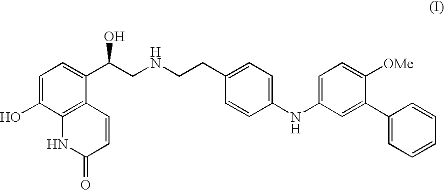

3. Crystalline N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate.

4. Crystalline 2,5-dichlorobenzenesulfonate salt of compound (I):

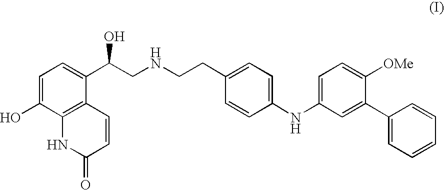

5. A compound as claimed in claim 1 which has a melting point onset measured by DSC (0.5° C.) in the range of 190-230° C.

6. A compound as claimed in claim 5 wherein the range is 219-223° C.

7. Crystalline N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylammonium 2,5-dichlorobenzenesulfonate characterized by an X-ray powder diffraction (XRPD) pattern including diffraction peaks at the following 2θ values: 11.6±0.1; 14.5±0.1; 23.2±0.1; 25.0±0.1; 27±0.1.

8. A process for preparing a compound of claim 1 which comprises contacting N-{2-[4-(3-phenyl-4-methoxyphenyl)aminophenyl]ethyl}2-hydroxy-2-(8-hydroxy-2(1H)-quinolinon-5-yl)ethylamine or a salt thereof with a source of 2,5-dichlorobenzenesulfonate ion and recovering the 2,5-dichlorobenzenesulfonate salt of claim 1.

9. A process according to claim 8 which comprises deprotecting a protected form of N-{2-[4-(3-phenyl-4-methoxyphenyl)aminophenyl]ethyl}-2-hydroxy-2-(8-hydroxy-2(1H)-quinolinon-5-yl)ethylamine, generating a protonated form of N-{2-[4-(3-phenyl-4-methoxyphenyl)aminophenyl]ethyl}-2-hydroxy-2-(8-hydroxy-2(1H)-quinolinon-5-yl)ethylamine and contacting said protonated form of N-{2-[4-(3-phenyl-4-methoxyphenyl)aminophenyl]ethyl}-2-hydroxy-2-(8-hydroxy-2(1 h)-quinolinon-5-yl)ethylamine with a source of 2,5-dicholorobenzenesulphonate ion.

10. A process according to claim 9 wherein said starting salt or protonated form of N-{2-[4-(3-phenyl-4-methoxyphenyl)aminophenyl]ethyl}-2-hydroxy-2-(8-hydroxy-2(1H)-quinolinon-5-yl)ethylamine is an acetate salt.

11. A process according to claim 8 wherein the 2,5-dichlorobenzenesulfonate salt of N-{2-[4-(3-phenyl-4-methoxyphenyl)aminophenyl]ethyl}-2-hydroxy-2-(8-hydroxy-2(1H)-quinolinon-5-yl)ethylamine is recovered in crystalline form.

12. A process according to claim 11 wherein crystallization is effected by contacting a solution of the 2,5-dichlorobenzenesulfonate salt with an antisolvent.

13. A process for obtaining crystalline 2,5-dichlorobenzenesulfonate salt of N-{2-[4-(3-phenyl-4-methoxyphenyl)aminophenyl]ethyl}-2-hydroxy-2-(8-hydroxy-2(1H)-quinolinon-5-yl)ethylamine which comprises recrystallising a compound as claimed in claim 1.

14. A process according to claim 13 wherein said a compound is dissolved in a solvent and crystallization effected by addition of an antisolvent.

15. A process according to claim 15 wherein the solvent is aqueous tetrahydrofuran and the antisolvent is water.

16. A pharmaceutical formulation comprising a compound according to claim 1 and a pharmaceutically acceptable carrier or excipient, and optionally one or more other therapeutic ingredients.

17. A method for the prophylaxis or treatment of a clinical condition in a mammal for which a selective β₂-adrenoreceptor agonist is indicated, which comprises administering a therapeutically effective amount of a compound according to claim 1.

18-19. (canceled)

20. A combination comprising a compound according claim 1 to and one or more other therapeutic ingredients.

21. A combination according to claim 21 wherein the other therapeutic ingredient is a PDE4 inhibitor or an anticholinergic or a corticosteroid.

22. A combination according to claim 20 wherein the one or more therapeutic agents is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

23. A method according to claim 17 wherein the mammal is a human.
